(19) United States
(12) Patent Application Publication
(10) Pub. No.: US 2026/0105598 A1
(43) Pub. Date: Apr. 16, 2026

(54) METHOD AND SYSTEM FOR SPINE LABELING IN MRI

(71) Applicant: GE Precision Healthcare LLC, Waukesha, WI (US)

(72) Inventors: Ashish Saxena, Bengaluru (IN); Dattesh Shanbhag, Bengaluru (IN); Chitresh Bhushan, Niskayuna, NY (US)

(73) Assignee: GE Precision Healthcare LLC, Waukesha, WI (US)

(21) Appl. No.: 18/917,702

(22) Filed: Oct. 16, 2024

(51) Int. Cl. G06T 7/00; G16H 30/40

(52) U.S. Cl. CPC G06T 7/0012; G16H 30/40; G06T 2207/10088; G06T 2207/30012

(57) ABSTRACT

A method and system for processing a magnetic resonance image of a spine includes receiving MR image data containing a plurality of spine images of a patient, including a spine image for each of a plurality of sagittal slices, and then processing each spine image with a deep learning (DL) model, wherein the DL model is trained to generate a plurality of labeled images for each spine image, wherein the plurality of labeled images are each labeled with a different predetermined set of spine level labels. The plurality of labeled images are combined into a labeled spine image for each of the plurality of slices, wherein each labeled spine image has a spine level label for each of a predetermined plurality of spine levels. A labeled MR image is then generated based on the labeled spine images.

### BACKGROUND

The present disclosure generally relates to systems and methods for magnetic resonance imaging (“MRI”). More particularly, the disclosure relates to systems and methods for performing calculations for automatically identifying and labeling spine levels in magnetic resonance (MR) images.

MRI is often used to obtain internal physiological information about a patient, including for brain imaging, spine imaging, cardiac imaging and imaging other sections or tissues within a patient's body (anywhere on the patient).

MRI uses the nuclear magnetic resonance (“NMR”) phenomenon to produce images. When a substance such as human tissue is subjected to a uniform magnetic field, such as the so-called main magnetic field (polarizing field B₀) generated by an MRI system, the individual magnetic moments of the nuclei in the tissue attempt to align with this B₀ field, but precess about it in random order at their characteristic Larmor frequency. If the substance, or tissue, is subjected to a magnetic field (excitation field B1) which is in the x-y plane and which is near the Larmor frequency, the net aligned moment, or “longitudinal magnetization”, Mz, may be rotated, or “tipped”, into the x-y plane to produce a net transverse magnetic moment Mt. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradients, sometimes referred to as readout gradients, vary according to the particular localization method being used. The resulting set of received signals are digitized and processed to reconstruct the image using reconstruction techniques.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect of the disclosure, a method for processing a magnetic resonance image of a spine includes receiving MR image data containing a plurality of spine images of a patient, including a spine image for each of a plurality of slices, and then processing each spine image with a deep learning (DL) model, wherein the DL model is trained to generate a plurality of labeled images for each spine image, wherein the plurality of labeled images are each labeled with a different predetermined set of spine level labels. The plurality of labeled images are combined into a labeled spine image for each of the plurality of slices, wherein each labeled spine image has a spine level label for each of a predetermined plurality of spine levels. A labeled MR image is then generated based on the labeled spine images.

In one embodiment, the plurality of labeled spine images generated for each spine image includes at least a first labeled image that includes only a first spine level label for a predetermined anchor level and a full labeled image that includes a spine level label for each of the predetermined plurality of spine level labels.

In another embodiment, each spine level label is located at a centroid of a vertebrae for each of the predetermined set of spine levels in each spine image.

In another embodiment, further comprising receiving one of a cervical designation or a lumber designation. In response to receiving the cervical designation the DL model generates at least a first labeled cervical image having an S1 label and a full labeled cervical image having an S1 label, an L5 label, an L4 label, an L3 label, an L2 label, an L1 label, and a T12 label. In response to receiving the lumbar designation, the DL model generates at least a first labeled lumbar image with a C1 label and a full labeled lumbar image with a C1 label, a C2 label, a C3 label, a C4 label, a C5 label, a C6 label, and a C7 label.

In another embodiment, further comprising creating the ML model by training a convolutional neural network to receive a spine image and generate a labeled image for each of the predetermined plurality of spine levels between a first assigned level and a last assigned level, wherein a first labeled image includes only a first spine level label, and wherein a full labeled image includes the spine level labels for each of the plurality of spine levels.

In another embodiment, wherein the DL model comprises a trained image segmentation model.

In another embodiment, wherein the DL model comprises a first U-Net trained to locate and label a centroid of a vertebrae for each of the predetermined sets of spine levels.

In another embodiment, wherein the DL model is a W-Net and comprises a second U-Net trained to refine the shape of the labels in each spine image.

In another embodiment, further comprising aligning the plurality of spine level labels across the plurality of slices in the labeled spine images to identify a 3D label volume for each of the predetermined plurality of spine levels.

In another embodiment, adjusting at least one of the spine level labels in at least one of the plurality of slices to make the 3D label volume for each of the predetermined plurality of spine levels contiguous across the plurality of slices.

In another embodiment, further comprising aligning the plurality of spine level labels across the plurality of slices in the labeled spine images to identify a plurality of 3D volumes that encompass the spine level labels, and then determining a distance between each of the plurality of 3D volumes. Where the distance between two 3D volumes of the plurality of 3D volumes is less than a threshold distance, the method further includes combining the two 3D volumes together to form a 3D label volume for one of the plurality of spine levels.

In another embodiment, further comprising processing the MR image data with an image segmentation model trained to identify a vertebrae mask labeling pixels associated with each vertebrae in the MR image data, comparing the vertebrae mask to the plurality of spine level labels to detect a missing label, and generating at least one spine level label to be added to the plurality of spine level labels based on the vertebrae masks.

In another embodiment, wherein missing label from the spine level labels is a spine level label for one of the predetermined plurality of spine levels.

In another embodiment, further comprising processing the MR image data with an image segmentation model trained to identify a vertebrae mask labeling pixels associated with each vertebrae in the MR image data and/or a disc mask labeling pixels associated with each disc in the MR image data, comparing the vertebrae mask and/or the disc mask to the plurality of spine level labels to detect a fused vertebrae, and generating a user prompt requesting clinician confirmation of the fused vertebrae.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a schematic diagram of an MRI system in accordance with an exemplary embodiment.

FIG. 2 is a flow chart illustrating an exemplary method of labeling an MR image of a spine according to one embodiment of the present disclosure.

FIG. 3 illustrates an exemplary trained DL model with inputs and outputs MR image of a spine according to one embodiment of the present disclosure.

FIG. 4 illustrates exemplary model architecture of a trained DL model according to one embodiment of the present disclosure.

FIGS. 5-6 illustrate exemplary steps and functions for labeling an MR image of a spine according to one embodiment of the present disclosure.

FIG. 7 is a flow chart illustrating another exemplary method of labeling an MR image of a spine according to embodiments of the present disclosure.

FIG. 8 is an exemplary labeled MR image exemplifying an output according to embodiments of the present disclosure.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of “top,” “bottom,” “front,” “rear,” “left,” “right,” “horizontal,” “vertical,” and “longitudinal” features and/or relative motion, e.g., movement “up” and “down,” is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally or alternatively, embodiments may be arranged in a different orientation such that “top” and “bottom” features are arranged horizontally relative to each other, for example in a “left-to-right” orientation.

The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

The present inventors have recognized that current methods and systems for automatically identifying and labeling spine levels in MR images are inaccurate and unreliable, often producing erroneous labels where spine levels are mislabeled, labeled multiple times, or vertebrae in the image are missed and thus not labeled at all. Sometimes artifacts or outliers are mislabeled as part of the spine image. Thus, the inventors have endeavored to develop improved systems and methods for automatically detecting and labeling spine levels in MR spine images.

The disclosed methods and system are configured to receive MR image data and to use a trained deep learning (DL) model to process the spine image for each of a plurality of slices, such as a plurality of 2D slice images, to generate a plurality of labeled images for each spine image at each slice. The DL model is configured to generate at least two labeled images for each image at each slice, wherein each of the at least two labeled images have a different predetermined set of spine level labels. The plurality of labeled images are then combined into a labeled spine image for each of the plurality of slices, wherein each labeled spine image has a spine level label for each of a predetermined plurality of spine levels, and a labeled MR image is generated based on the labeled spine images. For example, the labeled spine images may be 2D image slices. Alternatively or additionally, the disclosed methods and systems may be configured to receive image data that is 3D MRI data or 3D computed tomography (CT) data. Thereby, the DL model is configured to more robustly identify and generate spine level labels.

The plurality of labeled images generated for each original slice image includes at least two images with different spine level labels, such as a first labeled image with a first level labeled (e.g., the bottom lumbar spine level S1 or the top cervical spine level C1) and a full labeled image that includes spine level labels for each of the predetermined plurality of spine level labels that the DL is trained to identify. In some embodiments, the DL model is configured to generate a number of labeled images equal to the number of spine levels in the predetermined set of spine level labels. For example, the DL model may be trained to identify cervical levels, such as including C1-C7. In one embodiment, the DL model may be trained to generate seven images with each of the seven images containing a different predetermined set of spine level labels between C1 and C7, including a first image containing a C1 label, a second image containing a C1 label and a C2 label, a third image containing a C1 label, a C2 label, and a C3 label, and so on up to a seventh image containing seven labels for each of C1 through C7. Alternatively or additionally, the DL model may be trained to identify lumber levels, which in one embodiment may include S1 to T12, and generate seven images with each of the seven images containing a different predetermined set of spine level labels between S1 and T12 (e.g., a first image with just an S1 label, a Second image with an S1 label and an L1 label, and so on through a seventh image with labels for each of the S1 through T12 labels).

In some embodiments, the plurality of spine level labels across the plurality of slices in the labeled spine images are combined together and to identify a plurality of 3D volumes that encompass each of the spine level labels. For example, the plurality of 3D volumes may include one 3D volume for each of the predetermine plurality of spine levels, and wherein the depth of the 3D volume is based on the plurality of slices. The 3D volumes may be used as a processing tool to correct the labeling, including merging broken labels, filling in missing labels, identifying fused vertebrae, propagating spine level labels to additional levels, and other functions. In one example, the system and method are configured to determine a distance between each of the plurality of 3D volumes and assess the correctness of the labels based on the distances. Where the distance between two 3D volumes of the plurality of 3D volumes is less than a threshold distance, two 3D volumes may be combined together to form a 3D label volume for one of the plurality of spine levels. For example, the threshold distance may be a predetermined number of pixels or may be a distance measurement, such as millimeters.

Alternatively or additionally, the system may include one or more additional DL models trained to process the same MR image data and generate vertebrae masks and/or disc masks. Information from vertebrae masks and/or disc masks, such as each generated by trained image segmentation models, may be utilized to conduct further correction of the spine level labels. For example, the system may be configured to perform a comparison between the pixel locations in the mask(s) and those of the spine level labels to identify incorrect labels, fused vertebrae, and/or missed labels. In various implementations of such embodiments, missed levels may include unidentified levels in the predetermined plurality of spine levels and/or may include additional levels above or below the predetermined plurality of spine levels. Thereby, the system is configured to self-correct errors generated in the spine level labeling performed by the above-described DL model.

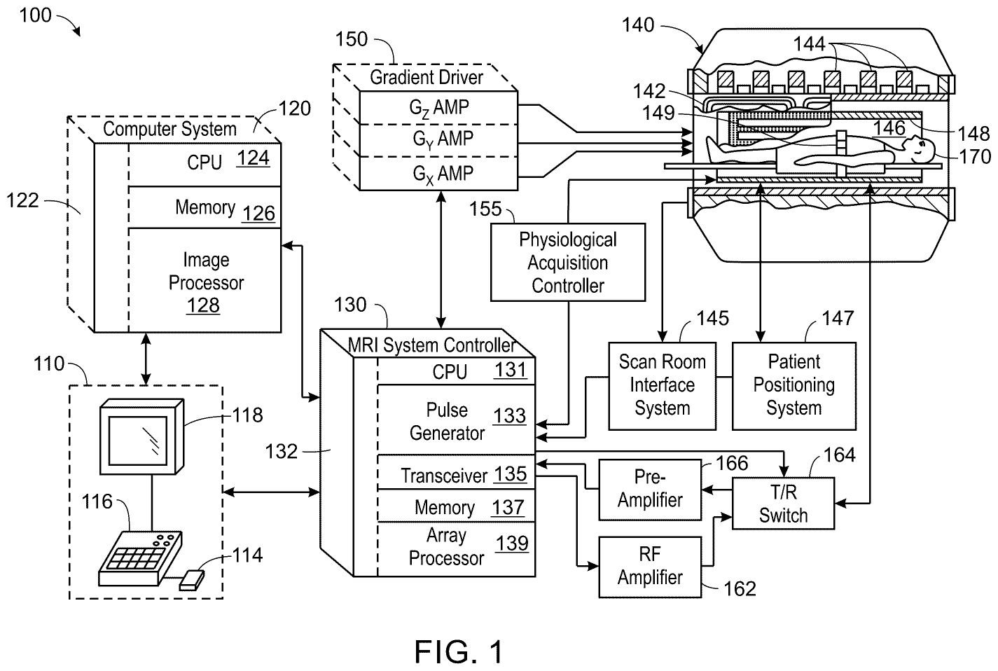

Referring to FIG. 1, a schematic diagram of an exemplary MRI system 100 is shown in accordance with an embodiment. The operation of MRI system 100 is controlled from an operator workstation 110 that includes an input device 114, a control panel 116, and a display 118. The input device 114 may be a joystick, keyboard, mouse, track ball, touch activated screen, voice control, or any similar or equivalent input device. The control panel 116 may include a keyboard, touch activated screen, voice control, buttons, sliders, or any similar or equivalent control device. The operator workstation 110 is coupled to and communicates with a computer system 120 that enables an operator to control the production and viewing of images on display 118. The computer system 120 includes a plurality of components that communicate with each other via electrical and/or data connections 122. The computer system connections 122 may be direct wired connections, fiber optic connections, wireless communication links, or the like. The components of the computer system 120 include a central processing unit (CPU) 124, a memory 126, which may include a frame buffer for storing image data, and an image processor 128. In an alternative embodiment, the image processor 128 may be replaced by image processing functionality implemented in the CPU 124. The computer system 120 may be connected to archival media devices, permanent or back-up memory storage, or a network. The computer system 120 is coupled to and communicates with a separate MRI system controller 130.

The MRI system controller 130 includes a set of components in communication with each other via electrical and/or data connections 132. The MRI system controller connections 132 may be direct wired connections, fiber optic connections, wireless communication links, or the like. The components of the MRI system controller 130 include a CPU 131, a pulse generator 133, which is coupled to and communicates with the operator workstation 110, a transceiver 135, a memory 137, and an array processor 139. In an alternative embodiment, the pulse generator 133 may be integrated into a resonance assembly 140 of the MRI system 100. The MRI system controller 130 is coupled to and receives commands from the operator workstation 110 to indicate the MRI scan sequence to be performed during a MRI scan. The MRI system controller 130 is also coupled to and communicates with a gradient driver system 150, which is coupled to a gradient coil assembly 142 to produce magnetic field gradients during a MRI scan.

The pulse generator 133 may also receive data from a physiological acquisition controller 155 that receives signals from a plurality of different sensors connected to an object or patient 170 undergoing a MRI scan, including electrocardiography (ECG) signals from electrodes attached to the patient 170. And finally, the pulse generator 133 is coupled to and communicates with a scan room interface system 145, which receives signals from various sensors associated with the condition of the resonance assembly 140. The scan room interface system 145 is also coupled to and communicates with a patient positioning system 147, which sends and receives signals to control movement of a table 171. The able 171 is controllable to move the patient in and out of the core 146 and to move the patient to a desired position within the core 146 for a MRI scan.

The MRI system controller 130 provides gradient waveforms to the gradient driver system 150, which includes, among others, G_{X}, G_{Y} and G_{Z} amplifiers. Each G_{X}, G_{Y} and G_{Z} gradient amplifier excites a corresponding gradient coil in the gradient coil assembly 142 to produce magnetic field gradients used for spatially encoding MR signals during a MRI scan. The gradient coil assembly 142 is included within the resonance assembly 140, which also includes a superconducting magnet having superconducting coils 144, which in operation, provides a homogenous longitudinal magnetic field B₀ throughout a core 146, or open cylindrical imaging volume, that is enclosed by the resonance assembly 140. The resonance assembly 140 also includes a RF body coil 148 which in operation, provides a transverse magnetic field B₁ that is generally perpendicular to B₀ throughout the core 146. The resonance assembly 140 may also include RF surface coils 149 used for imaging different anatomies of a patient undergoing a MRI scan. The RF body coil 148 and RF surface coils 149 may be configured to operate in a transmit and receive mode, transmit mode, or receive mode.

An object or patient 170 undergoing a MRI scan may be positioned within the core 146 of the resonance assembly 140. The transceiver 135 in the MRI system controller 130 produces RF excitation pulses that are amplified by an RF amplifier 162 and provided to the RF body coil 148 and RF surface coils 149 through a transmit/receive switch (T/R switch) 164.

As mentioned above, RF body coil 148 and RF surface coils 149 may be used to transmit RF excitation pulses and/or to receive resulting MR signals from a patient undergoing a MRI scan. The resulting MR signals emitted by excited nuclei in the patient undergoing a MRI scan may be sensed and received by the RF body coil 148 or RF surface coils 149 and sent back through the T/R switch 164 to a pre-amplifier 166. The amplified MR signals are demodulated, filtered and digitized in the receiver section of the transceiver 135. The T/R switch 164 is controlled by a signal from the pulse generator 133 to electrically connect the RF amplifier 162 to the RF body coil 148 during the transmit mode and connect the pre-amplifier 166 to the RF body coil 148 during the receive mode. The T/R switch 164 may also enable RF surface coils 149 to be used in either the transmit mode or receive mode.

The resulting MR signals sensed and received by the RF body coil 148 are digitized by the transceiver 135 and transferred to the memory 137 in the MRI system controller 130.

A MR scan is complete when an array of raw k-space data, corresponding to the received MR signals, has been acquired and stored temporarily in the memory 137 until the data is subsequently transformed to create images. This raw k-space data is rearranged into separate k-space data arrays for each image to be reconstructed, and each of these separate k-space data arrays is input to the array processor 139, which operates to Fourier transform the data into arrays of image data.

The array processor 139 uses a known transformation method, most commonly a Fourier transform, to create images from the received MR signals. These images are communicated to the computer system 120 where they are stored in memory 126. In response to commands received from the operator workstation 110, the image data may be archived in long-term storage or it may be further processed by the image processor 128 and conveyed to the operator workstation 110 for presentation on the display 118.

In various embodiments, the components of computer system 120 and MRI system controller 130 may be implemented on the same computer system or a plurality of computer systems.

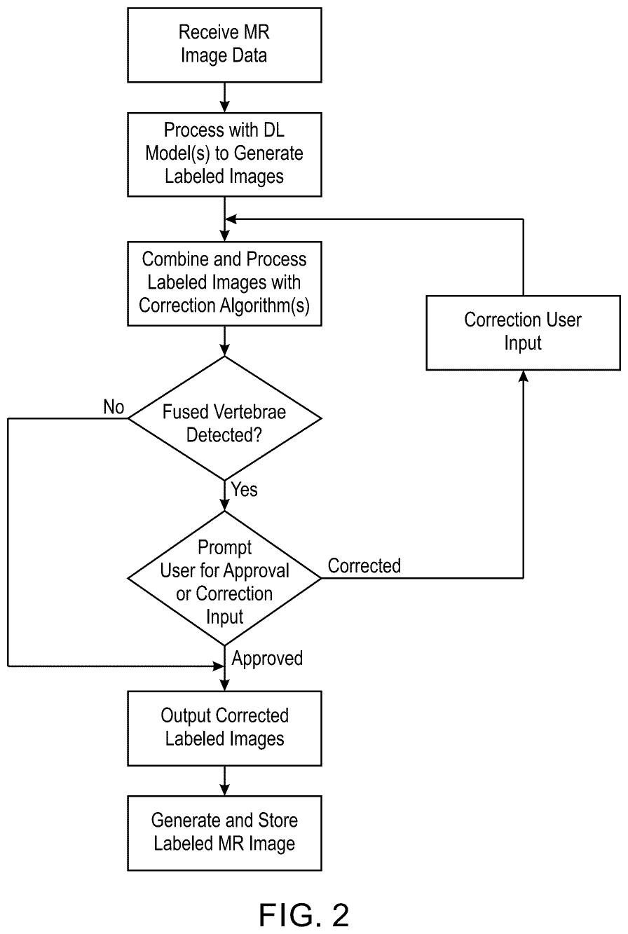

FIGS. 2 through 4 exemplify method steps and system architectures for identifying spine level labels according to embodiments of the present disclosure. FIG. 2 depicts an exemplary method 200 of processing an MR image of a spine to generate a labeled MR image containing accurate spine level labels. MR image data is received at step 202. The MR image data includes at least one 2D image representing at least one slice, as is standard in MR imaging. In many implementations, the MR data includes a plurality of images, each representing one of a plurality of slices, such as sagittal 2D slices each including an image of the patient's spine along a sagittal plane. The MR image data is processed at step 204 with at least one DL model trained to generate labeled images wherein one or more spine levels are labeled within the sagittal slice images of the patient's spine. For example, the DL model is trained to receive each 2D sagittal slice spine image, wherein the plurality of labeled images are each labeled with a different predetermined set of spine level labels. The outputted labeled images are combined and processed with one or more correction algorithms at step 206, wherein the correction algorithms are configured to merge broken labels, identify and remove outliers, and/or fix missing labels.

In some embodiments, the correction algorithm is also configured to identify and correct fused vertebrae and/or other physiological abnormalities captured in the image. For example, the correction algorithm may be configured to generate 3D label volumes for each of the labels and to refine the spine level labels based on the 3D volumes. Alternatively or additionally, the correction algorithm may include one or more trained DL models configured to process the MR image data and/or the labeled images to generate vertebrae masks and/or disc masks, and to uses those masks to correct and refine the spine level labels in the labeled images. The system may also be configured to prompt a user for input confirming and/or correcting the labels and to utilize the user input as additional input to the correction algorithm(s), in addition to the labeled images.

In the depicted embodiment, the correction algorithm is configured to identify and label fused vertebrae, and step 208 is performed identifying whether any fused vertebrae were detected by the correction algorithms. If so, step 210 is performed to prompt a user to review and provide input approving of the automatically generated labels (including the fusion identification) or provide input correcting one or more of the spinel level labels and/or fusion identification(s). User input is received, which may be either an approval or a correction. Where a correction user input is received, such correction input is provided at step 212 to the correction algorithm(s). The user input may be in any number of forms, such as moved labels and/or a selection of pixels representing the location of fused vertebrae or representing a disc or another point between two labels.

Steps 206 and 208 may be repeated utilizing the correction user input as an additional input to the correction algorithm until user approval input is received at step 210. The corrected labeled images are then outputted at step 214. The corrected images, which include a plurality of 2D sagittal images each containing spine level labels for the predetermined plurality of spine levels are stored at step 216 and/or outputted as a labeled MR image.

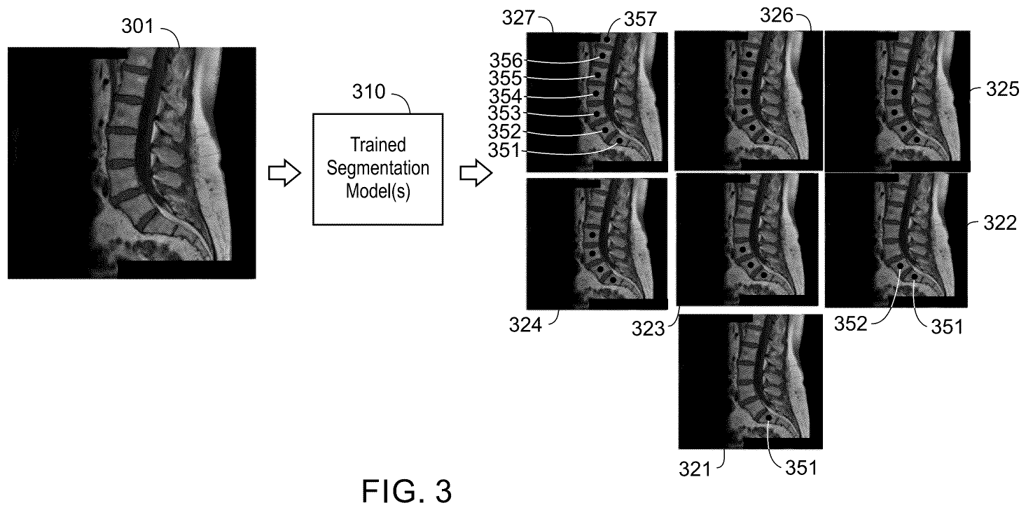

FIG. 3 represents input, outputs, and structure of an exemplary DL model configured according to the present disclosure. The DL model is a trained segmentation model 310 trained to locate and label a predetermined plurality of spine levels in the MR images, which may be a predetermined set of lumbar levels and/or a predetermined number of cervical levels. The DL model 310 receives a 2D spine image 301 for each sagittal slice in the MR image data and to generate a plurality of labeled images 321-327 therefrom. As is well known, MR image data contains a plurality of images, each representing a different slice depth in the patient. Here, the MR image data is of a patient's spine, which is generally taken at a plurality of sagittal slices across the width of the patient. Thus, each slice image captures multiple vertebrae, which may be of the lumbar section of the spine or the cervical section of the spine, or may be an image capturing both the lumbar and cervical regions, such as the entire spine of the patient. Each of the plurality of spine images in the MR image data is provided to the trained DL model, which is trained to produce multiple labeled images for each inputted spine image 301.

As shown in the example, the model is trained to produce the plurality of labeled images 321-327 for each inputted spine image comprising a slice of the MR image data. Each of the plurality of slice images are likewise processed, and thus multiple sets of labeled images 321-327 are generated for the inputted MR image data. Each of the plurality of labeled images 321-327 is labeled with a different predetermined set of spine level labels, including a first labeled image that includes only a first spine level label, such as for a predetermined anchor level at the top or bottom of the spine, and a full labeled image 327 that includes a spine level label for each of the predetermined plurality of spine levels that the DL model 310 is configured to label.

FIG. 3 shows an embodiment where the DL model 310 is configured to generate seven labeled images 321-327, including one for each of the seven predetermined spine levels that the DL model is trained to identify (which in this example is S1-T12). The seven labeled images 321-327 each contain a spine level label for the anchor level, which may be S1 for lumbar images and C1 for cervical images. The first spine level image 321 comprises a first spine level label 351, and then each successive image includes an additional spine level label up to the last image, the full labeled image, which contains spine level labels for all of the plurality of spine levels that the DL model 310 is trained to identify. Thus, the second labeled image 322 includes the first spine level label 351 and the second spine level label 352; the third labeled image 323 includes the first spine level label 351, the second spine level label 352, and the third spine level label 353, and so on up to full labeled image. Here, the full labeled image is the seventh labeled image 327 that includes the first spine level label 351, the second spine level label 352, the third spine level label 353, the fourth spine level label 354, the fifth spine level label 355, the sixth spine level label 356, and the seventh spine level label 357. Here, the spine image captures the lumbar region of the patient and the predetermined plurality of spine levels are S1, L5, L4, L3, L2, L1, and T12, which correspond respectively with the illustrated spine level labels 351-357.

The DL model 310 may be any of the various types of image segmentation models trained to label the predetermined plurality of spine levels in the MR images, which may be a predetermined set of cervical levels and/or a predetermined set of lumbar levels. For example, the DL model 310 may be a U-Net architecture, which is a convolutional network containing successive layers utilizing upsampling operators. In some embodiments, the DL model 310 may comprise multiple U-Net architectures concatenated together. In one example, the DL model is a W-Net architecture comprising two U-Net architectures, the first U-Net acting as an encoder that outputs a segmentation of the spine image and the second U-Net acting as a decoder that reconstructs the image from the output of the first U-Net.

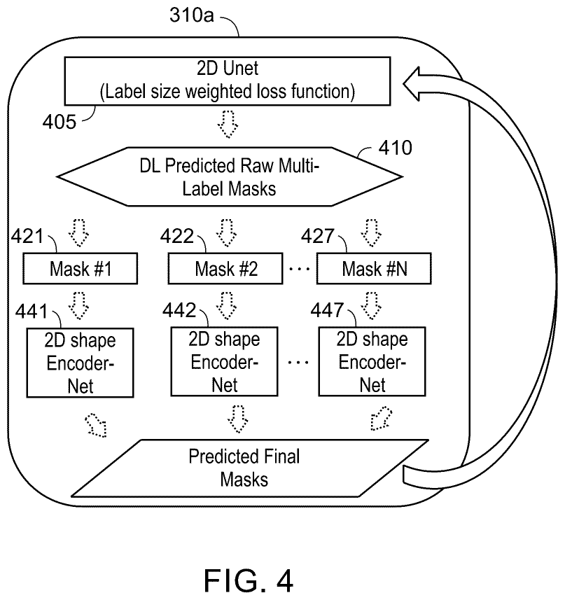

FIG. 4 illustrates one such embodiment, which is an exemplary architecture of a trained DL model configured to generate a plurality of labeled spine images for each 2D sagittal slice spine image, like those illustrated in FIG. 3. Here, the trained DL model 301a is a W-Net architecture with size-weighted dice and a per-mask shape encoder. The first U-Net 405 is a U-Net trained to locate and label a centroid of a vertebra for each of the predetermined set of spine levels, and to generate the plurality of labeled images (e.g., 321-327) containing the spine level labels. The first U-Net 405 provides the predicted label masks as output, such as containing each of the predetermined sets of spine level labels 351-357 shown and described in FIG. 2.

The output from the first U-Net 405 is provided to second U-Net 410 that is trained to receive the output of the first U-Net and act as a decoder providing a plurality masks 421-427, including one mask for each of the predetermined set of spine level labels in each of the plurality of spine images 221-227. Thus, the number of outputs from the second U-Net 410, or decoder U-Net, aligns with the number of labeled images in the plurality of labeled images generated by the model 301a. The decoder U-Net 410 may be configured such that each of the output channels for each of the masks 421-427 are equally weighted regardless of the number of spine level labels in that channel. Thus, the first channel configured to output the first mask 421 for the first labeled image, which only has one spine level label, is weighted the more per labeled pixel than the last channel configured to output the mask 427 for the full labeled image, such that the focus of the model 410 is balanced evenly on all output channels. In other embodiments, the model 310a architecture may comprise multiple decoder U-Nets 410, such as one decoder U-Net per mask 421-427, wherein each decoder U-Net is trained to generate one of the respective masks 421-427.

Each outputted mask 421-427 is provided to a respective shape encoder 441-447 configured to refine the shape of the labels in each spine image. The shape encoders 421-427 may be an additional U-Net configured to refine the shape of the group of pixels identified in each spine level label, such as to adjust each spine level label such that it includes a certain number of labels in a certain shape. The outputs of the shape encoders 441-447 are combined to generate the predicted final masks that are outputted by the DL model 301a.

In some embodiments, the system may comprise multiple DL models 310a, wherein each DL model is trained to identify a different predetermined plurality of spine levels. For example, the system may comprise a lumbar model configured to identify a predetermined plurality of lumbar spine levels and a cervical model configured to identify a predetermined set of cervical spine levels. Other embodiments may include a thoracic model configured to identify thoracic levels, which may be in combination with cervical and/or lumber levels. Alternatively, one model 310a may be configured to identify all the different sets of predetermined pluralities of spine levels.

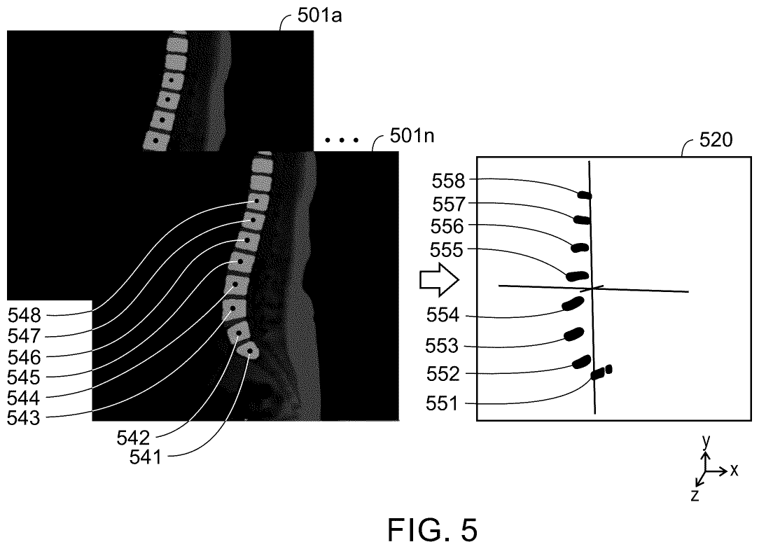

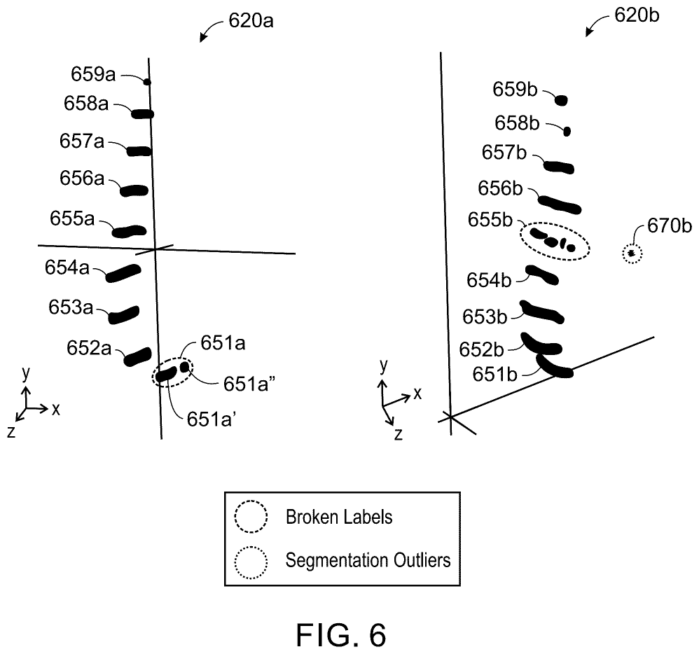

The output of the DL model 310 is provided to at least one correction algorithm in executed as part of a correction module configured to correct the spine level labels across the plurality of labeled images, including to merge broken labels, identify and remove outliers, and/or fix missing labels. The correction module includes software instructions stored on a non-transitory computer-readable medium and executable in order to process the various spine image data described herein and to correct the spine level label information generated by the DL model(s) 310, 410. For example, the correction module and/or the DL model(s) 310, 410 may be stored and executed within the computer system 122 described herein. The correction module may include one or more trained DL models, such as image segmentation models or other image processing models described herein. In some embodiments, the correction module is also configured to identify and correct fused vertebrae and/or other physiological abnormalities captured in the image. For example, the correction module may be configured to generate 3D label volumes for each of the labels and to refine the spine level labels based on the 3D volumes. FIGS. 5-6 illustrate exemplary steps including generating 3D label volumes based on spine level labels across the plurality of labeled slice images generated based on the labeled spine images. The correction module may comprise programming configured to merge some or all of the labeled images for each slice into a merged labeled image for each slice. In one embodiment, the first and last labeled images for each slice (e.g., labeled images 321 and 327 in the example in FIG. 3) are merged together to create the merged labeled image for that slice. The merged labeled image includes all of the labels for the predetermined plurality of spine levels since the last image (i.e., the full labeled image) includes all of the spine level labels. Since both the first labeled image and the last labeled image contain a spine level label for the anchor level, the merged labeled image will have a double weighted spine level label for the anchor level (e.g., whether it is S1 for the lumbar image or C1 for the cervical image). The anchor level can thus be identified accordingly. In other embodiments a different subset of the plurality of labeled images (e.g., labeled images 321-327 in the example in FIG. 3) may be utilized to generate the merged labeled image for each slice. Since all of the labeled images contain a spine level label for the anchor level, that label will always have the highest weight in the merged labeled image.

The merged labeled images, one for each of the slices in the MR image data, are then utilized to generate 3D label volumes for each of the spine levels being labeled. FIG. 5 illustrates this concept, where the plurality of merged labeled images 501a-501n are aligned such that a 3D label volume 551-558 can be identified for each of the predetermined plurality of spine levels, where the 3D label volumes are preferably contiguous across the plurality of slices. Here, the merged labeled images 501a-501n for the plurality of slices (e.g., n slices) each contain eight spine level labels 541-548. The spine level labels 541-548 across all of the aligned merged labeled images 501a-501n are unified into a set of 3D label volumes 551-558, including a 3D label volume 551-558 for each of the spine level labels 541-548 in the merged images. The shape of the 3D volumes 551-558 is thus dictated by the spine level labels. For example, where the spine level labels 541-548 each comprise groups of pixels arranged in a circle, the 3D volumes 551-558 will be roughly cylindrical in shape running approximately the width of the respective vertebrae as captured in the various slices in the MR image data.

The 3D label volumes are identified with respect to a 3D graph 520, including a x dimension (e.g., the frequency dimension of the MR image data), y dimension (e.g., the phase dimension of the MR image data), and z dimension (e.g., the slice dimension of the MR image data). The plotted 3D volumes may be processed to identify and correct issues with the spine level labels generated by the DL model, such as to merge broken labels, identify and remove outliers, and/or fill in missing labels. FIG. 6 shows two different 3D graphs 620a and 620b of 3D label volumes illustrating exemplary problems that the correction module is configured to fix.

The 3D labels are analyzed to identify broken labels and outliers and to correct for those errors. The 3D graph 620a shows a set of 3D label volumes 651a-659a for each of nine spine levels. The lowest 3D label volume 651a is a broken label, where the 3D label volume 651a is initially broken into two parts 651a′ and 651a″. This may be due to a missing spine level label in the merged labeled images for one or a few of the slices at the location of the break. The correction module may be configured to identify a broken label, such as based on the distances between the 3D volumes 651a′ and 651a″ and/or the respective locations of the volumes with respect to the x and/or y axes. For example, if two 3D volumes are less than a threshold distance apart, the 3D volumes may be combined together into a single 3D label volume for a single spine level. The threshold distance may vary, such as depending on the levels being imaged and/or the scale of the image. To provide just one example, the threshold distance may be 30 mm or 40 mm, or a corresponding number of pixels. In graph 620a, the 3D volumes 651a′ and 651a″ are less than the threshold distance apart, and thus they are joined together as a single 3D label volume by filling in the intervening pixels, e.g., interpolating between the two 3D volumes 651a′ and 651a″. This will add and/or adjust the spine level labels in each of the impacted merged labeled images for corresponding slices.

Alternatively or additionally, the broken labels may be identified by comparing the 3D label volumes to vertebrae mask volumes generated based on vertebrae masks for each of the slices, which are described in more detail herein. For example, where two 3D volumes, such as 651a′ and 651a″, appear in one 3D vertebrae mask volume, then those 3D volumes may be merged into a single 3D label volume, as just described.

The same process can be performed for the 3D label volumes shown in the graph 620b, which shows a set of 3D label volumes 651b-659b for each of nine spine levels. The 3D label volume 655b illustrates another broken label that can be fixed by the correction algorithm, such as based on the threshold distance assessment and/or comparison to a vertebrae mask volume. Graph 320b also illustrates a segmentation outlier, where a 3D volume 670b is not associated with any spine level and should not have been identified. The segmentation outlier may be identified based on various parameters, such as magnitude of the volume, relative location and/or distance from other 3D volumes, etc. For example, a 3D volume (e.g., 370b) may be labeled as a segmentation outlier and thus removed from the 3D label volumes and from the labeled images if it meets certain criteria—e.g., it is less than a threshold magnitude in size (e.g. less than a threshold volume or a threshold number of pixels), and/or it is not aligned with the other 3D label volumes 651b-659b in along the x and/or y axes, and/or where it is greater than a threshold distance from any other 3D spine level label.

In addition to the exemplified broken labels and/or erroneous labels, the correction module may be configured to split erroneously joined labels and/or to propagate missing labels. In some embodiments, the correction algorithm may include one or more trained DL models configured to process the MR image data and/or the labeled images to generate vertebrae masks and/or disc masks, and to uses those masks to correct and refine the spine level labels in the labeled images. For example, an image segmentation model may be trained to generate a vertebrae mask labeling pixels associated with each vertebrae in the MR image data. Alternatively or additionally, the correction module may comprise an image segmentation module trained to generate a disc mask labeling pixels associated with each disc in the MR image data. The 3D label volumes may be compared with the disc mask and/or the vertebrae mask to detect a split label or a missing label, or to propagate the spine level labels to additional spine levels above or below the predetermined plurality of spine levels that the DL model 310, 410 is trained to identify.

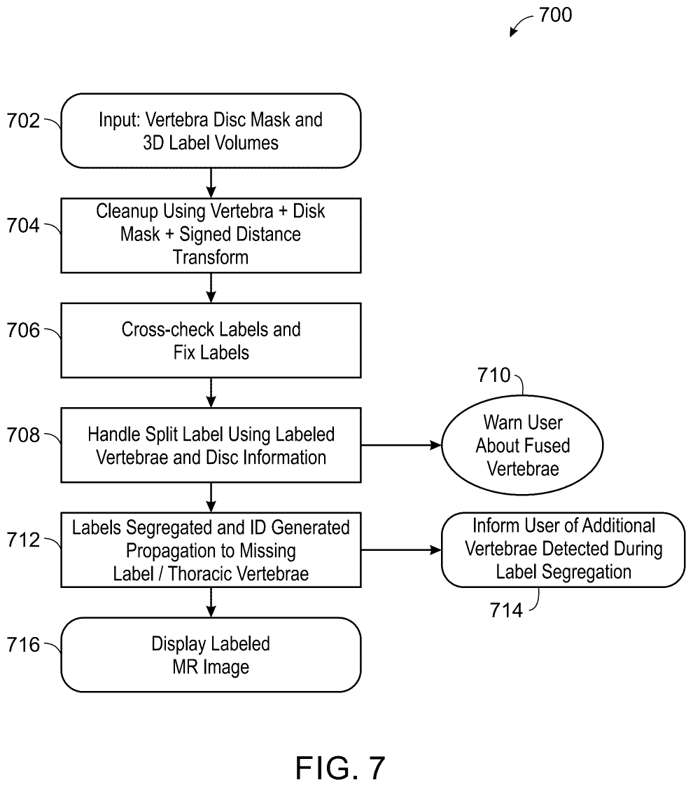

FIG. 7 exemplifies method 700 that comprises steps for correcting spine level labels, including steps and processes for correcting the labels using disc mask and/or vertebrae mask information, as well as for obtaining user input to revise and/or validate the spine level labels. Multiple inputs are received at step 702, including the 3D label volumes (and/or the corrected labeled images for the plurality of slices), a vertebrae mask, and a disc mask. Step 704 is executed to clean up the 3D label volumes by comparison to the vertebrae and disc masks, such as to identify and remove outliers and otherwise remove segmentation errors that are outside the region of interest. In one embodiment, spine region may be identified in the image data based on the disc mask and the vertebrae mask. Each of the spine level labels are then assessed to make sure that each label is within the spine region and/or within a threshold distance of the spine region. If any label is not within the spine region, then it is determined to be an outlier and is removed.

Step 706 is performed to cross-check the labels between the 3D label volumes (and/or the corrected labeled images for the plurality of slices) and one or more of the disc masks and the vertebrae masks, and then to fix labels based on the result of the comparison, such as to fill in missing labels. For example, the spine level labels for the 3D volumes may be overlayed with the vertebrae mask. If the 3D volumes are labeled correctly, each vertebra mask (or a 3D volume generated therefrom) should align or intersect with one 3D label volume and the respective labels should be for the same spine level. If they are not, then the differences are rectified to correct an error(s) in the spine level labels.

Step 708 is performed to identify a fused vertebrae based on a comparison of the 3D label volumes and the vertebrae masks and/or the disc masks. For example, if each 3D label volume is not separated by a disc based on the disc mask, or if any 3D volume intersects two vertebrae masks, then it may be determined that the 3d label volume spans over two vertebrae that are likely fused and needs to be split into two labels. An output is generated at step 710 to notify the user of the detected fused vertebra. In some embodiments, the correction module may be configured to prompt a user for input, such as input approving/confirming the fused vertebra recognition or providing a correction. The correction user input may then be utilized by the correction module to reprocess and revise the spine level labels, as described above.

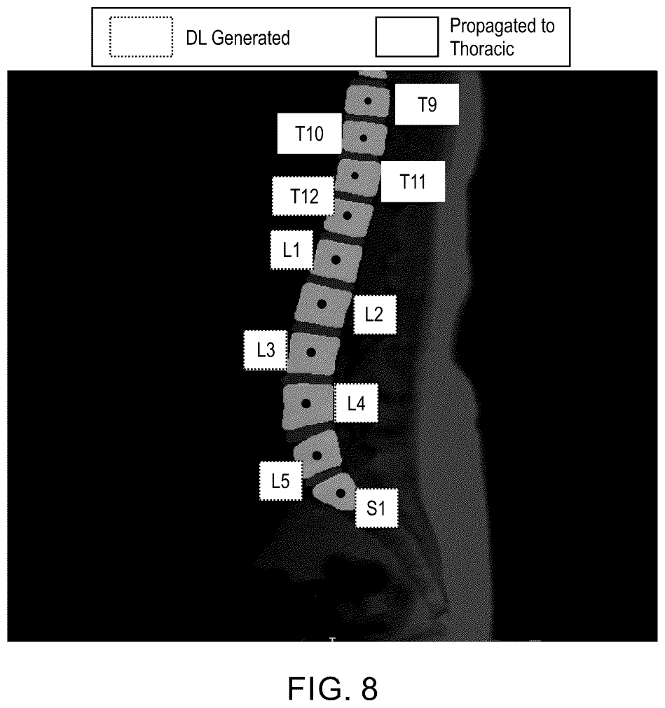

Step 712 is executed to identify missing labels based on a comparison of the 3D label volumes and the vertebrae masks and/or the disc masks. For example, where a vertebra mask for one vertebra does not intersect with any 3D label volume, then a missed label is identified and added. The missed label may be above or below the predetermined plurality of spine levels that the DL model 310, 410 is trained to detect. Accordingly, the disc mask and/or vertebrae mask information may be utilized to propagate the spine level labels to additional levels not labeled by the model and thus not included in the predetermined plurality of levels that the model is trained to identify, which will be counted and labeled in order starting with the closest labeled spine level in the 3D label volume. FIG. 8 illustrates an exemplary output of such a method, showing a labeled 2D image wherein each of a predetermined plurality of lumbar spine levels are labeled, and wherein the level labels are propagated up to label additional thoracic spine levels above the predetermined lumbar spine levels labeled by the trained DL model. The 2D images for each of the slices in the MR image data would be labeled accordingly.

An alert is provided to the user at step 714 regarding the additional detected and propagated labels. In some embodiments, this could include location of an abnormality, such as an L6 vertebra (which can be detected in a small minority of patients). The alert may also include notice of such an abnormality. The revised labeled images may be presented to the user as part of the alert. In some embodiments, user input may be requested or required and such user input may be utilized to affirm or revise the labeling, as is described above. The final labeled MR image is then stored and outputted and/or displayed at step 716. FIG. 8 depicts one slice of an exemplary labeled image.

In various embodiments, any suitable computer readable media can be used for storing instructions for performing functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

This written description uses examples to disclose the invention(s), including the best mode, and also to enable any person skilled in the art to make and use the invention(s). Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention(s) is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

1. A method for processing a magnetic resonance image of a spine, the method comprising:
receiving MR image data containing a plurality of spine images of a patient, including a spine image for each of a plurality of sagittal slices;
processing each spine image with a deep learning (DL) model, wherein the DL model is trained to generate a plurality of labeled images for each spine image, wherein the plurality of labeled images are each labeled with a different predetermined set of spine level labels;
combining the plurality of labeled images into a labeled spine image for each of the plurality of sagittal slices, wherein each labeled spine image has a spine level label for each of a predetermined plurality of spine levels; and
generating a labeled MR image based on the labeled spine images.

2. The method of claim 1, wherein the plurality of labeled images generated for each spine image includes at least a first labeled image that includes only a first spine level label and a full labeled image that includes the first spine level label and additional spine level labels for each of the predetermined plurality of spine levels.

3. The method of claim 1, wherein each spine level label is located at a centroid of a vertebrae in the spine image.

4. The method of claim 1, further comprising receiving one of a cervical designation or a lumbar designation;
wherein in response to receiving the cervical designation the DL model generates at least a first labeled cervical image having an S1 label and a full labeled cervical image having an S1 label, an L5 label, an L4 label, an L3 label, an L2 label, an L1 label, and a T12 label; and
wherein in response to receiving the lumbar designation, the DL model generates at least a first labeled lumbar image with a C1 label and a full labeled lumbar image with a C1 label, a C2 label, a C3 label, a C4 label, a C5 label, a C6 label, and a C7 label.

5. The method of claim 1, further comprising creating the DL model by training a convolutional neural network to receive the spine image and generate the labeled image for each of the predetermined plurality of spine levels between a first assigned level and a last assigned level, wherein a first labeled image includes only a first spine level label, and wherein a full labeled image includes the first spine level label and additional spine level labels for each of the predetermined plurality of spine levels.

6. The method of claim 1, wherein the DL model comprises at least a first U-Net trained to locate and label a centroid of a vertebrae for each of the predetermined sets of spine levels.

7. The method of claim 6, wherein the DL model is a W-Net and comprises a second U-Net trained to refine the shape of the labels in each spine image.

8. The method of claim 1, further comprising aligning the spine level labels across the plurality of sagittal slices in the labeled spine images to identify a 3D label volume for each of the predetermined plurality of spine levels.

9. The method of claim 8, adjusting at least one of the spine level labels in at least one of the plurality of sagittal slices to make the 3D label volume for each of the predetermined plurality of spine levels contiguous across the plurality of sagittal slices.

10. The method of claim 1, further comprising:
aligning the spine level labels across the plurality of sagittal slices in the labeled spine images to identify a plurality of 3D volumes that encompass the spine level labels;
determining a distance between each of the plurality of 3D volumes;
where the distance between two 3D volumes of the plurality of 3D volumes is less than a threshold distance, combining the two 3D volumes together to form a 3D label volume for one of the plurality of spine levels.

11. The method of claim 1, further comprising:
processing the MR image data with an image segmentation model trained to identify a vertebrae mask labeling pixels associated with each vertebrae in the MR image data;
comparing the vertebrae mask to the spine level labels to detect a missing label; and
generating at least one spine level label to be added to the predetermined plurality of spine level labels based on the vertebrae masks.

12. The method of claim 11, wherein missing label from the spine level labels is a spine level label for one of the predetermined plurality of spine levels.

13. The method of claim 1, further comprising:
processing the MR image data with an image segmentation model trained to identify a vertebrae mask labeling pixels associated with each vertebrae in the MR image data and/or a disc mask labeling pixels associated with each disc in the MR image data;
comparing the vertebrae mask and/or the disc mask to the spine level labels to detect a fused vertebrae; and
generating a user prompt requesting clinician confirmation of the fused vertebrae.

14. A magnetic resonance imaging (MRI) system comprising:
a magnet system configured to generate a polarizing magnetic field about at least a portion of a subject arranged in the MRI system;
a plurality of gradient coils configured to apply gradient pulses to the polarizing magnetic field;
a radio frequency (RF) system configured to apply an RF field to the subject and to acquire magnetic resonance (MR) image data therefrom;
a processing device; and
a memory storage device comprising instructions executable by the processing device to:
receive MR image data containing a plurality of spine images of a patient, including a spine image for each of a plurality of slices;
process each spine image with a deep learning (DL) model, wherein the DL model is trained to generate a plurality of labeled images for each spine image, wherein the plurality of labeled images are each labeled with a different predetermined set of spine level labels;
combine the plurality of labeled images into a labeled spine image for each of the plurality of slices, wherein each labeled spine image has a spine level label for each of a predetermined plurality of spine levels; and
generate a labeled MR image based on the labeled spine images.

15. The system of claim 14, wherein the plurality of labeled spine images generated for each spine image includes at least a first labeled image that includes only a first spine level label for a predetermined anchor level and a full labeled image that includes a spine level label for each of the predetermined plurality of spine levels.

16. The system of claim 14, wherein each spine level label is located at a centroid of a vertebrae for each of the predetermined set of spine levels in each spine image.

17. The system of claim 14, wherein the instructions executable by the processing device are further executable to:
align the spine level labels across the plurality of slices in the labeled spine images to identify a plurality of 3D volumes that encompass the spine level labels and process the plurality of 3D volumes to correct the spine level labels.

18. The system of claim 17, wherein the instructions executable by the processing device are further executable to:
determine a distance between each of the plurality of 3D volumes; and
where the distance between two 3D volumes of the plurality of 3D volumes is less than a threshold distance, combine the two 3D volumes together to form a 3D label volume for one of the plurality of spine levels.

19. The system of claim 14, wherein the instructions executable by the processing device are further executable to:
process the MR image data with an image segmentation model trained to identify a vertebrae mask labeling pixels associated with each vertebrae in the MR image data;
compare the vertebrae mask to the spine level labels to detect a missing label; and
generate at least one spine level label to be added to the predetermined plurality of spine level labels based on the vertebrae masks.

20. The system of claim 14, wherein the instructions executable by the processing device are further executable to:
process the MR image data with an image segmentation model trained to identify a vertebrae mask labeling pixels associated with each vertebrae in the MR image data and/or a disc mask labeling pixels associated with each disc in the MR image data;
compare the vertebrae mask and/or the disc mask to the spine level labels to detect a fused vertebrae;
generate a user prompt requesting clinician confirmation of the fused vertebrae.
